(19) United States
(12) Patent Application Publication
(10) Pub. No.: US 2002/0156460 A1
(43) Pub. Date: Oct. 24, 2002

(54) Microcatheter with improved distal tip and transitions

(75) Inventors: Ting Tina Ye, San Jose, CA (US); Rhoda M. Santos, Newark, CA (US); Elaine Lim, Fremont, CA (US); Mai Xuan Tran, San Jose, CA (US); Hanh Doan, Milpitas, CA (US); Simon Ngoc Huu Nguyen, San Jose, CA (US)

Correspondence Address: Robert E. Atkinson, CROMPTON, SEAGER & TUFTE, LLC, 331 Second Avenue South, Suite 895, Minneapolis, MN, 55401-2246, US

(73) Assignee: SciMed Life Systems, Inc

(21) Appl. No.: 09/839,065

(22) Filed: Apr. 20, 2001

(51) Int. Cl. A61M 25/00

(52) U.S. Cl. 604/534; 604/526; 156/123

(57) ABSTRACT

The present invention relates generally to intravascular catheters for performing medical procedures. An intravascular catheter is disclosed comprising a shaft, the shaft comprising an inner liner having a distal end, a distal tip; a second layer disposed over the inner liner, the second layer extending from the proximal end of the shaft to a distal terminus; a third layer disposed over the second layer; and a fourth layer disposed over the third layer, the fourth layer including a proximal end and a distal end. In addition, a method of manufacturing a catheter according to a preferred embodiment of the invention is disclosed.

### FIELD OF THE INVENTION

The present invention relates generally to intravascular catheters for performing medical procedures. More particularly, the present invention relates to intravascular catheters with improved shaft and distal tip designs.

### BACKGROUND OF THE INVENTION

Intravascular catheters are used in a wide variety of relatively non-invasive medical procedures. Such intravascular catheters may be used for diagnostic or therapeutic purposes. Generally, an intravascular catheter allows a physician to remotely perform a medical procedure by inserting the catheter into the vascular system of the patient at a location that is easily accessible and thereafter navigating the catheter to the desired target site. By this method, virtually any target site in the patient's vascular system may be remotely accessed, including the coronary, cerebral, and peripheral vasculature.

Typically, the catheter enters the patient's vasculature at a convenient location such as a blood vessel in the neck or near the groin. Once the distal portion of the catheter has entered the patient's vascular system, the physician may urge the distal tip forward by applying longitudinal forces to the proximal portion of the catheter. Frequently the path taken by a catheter through the vascular system is tortuous, requiring the catheter to change direction frequently. In some cases, it may even be necessary for the catheter to bend ninety degrees or more. In order for the catheter to navigate a patient's tortuous vascular system, it is desirable that intravascular catheters be very flexible, particularly near the distal end.

The distance between the access site and the target site is often in excess of 100 cm. The inside diameter of the vasculature at the access site is often less than 2 cm, and the inside diameter of the vasculature at the target site is often less than 0.5 cm. Accordingly, intravascular catheters must be relatively long and thin. Furthermore, in order to navigate through the patient's tortuous vascular system, intravascular catheters must be very flexible. It is also desirable that intravascular catheters be relatively soft in order to minimize the probability of damaging vascular tissue.

Intravascular catheters typically have a radiopaque portion and are guided through the patient's vascular system with the assistance of x-ray fluoroscopy. In this manner, a physician may manipulate the proximal end of the catheter and fluoroscopically monitor the corresponding movement of the distal end of the catheter. As such, it is desirable that intravascular catheters be sufficiently radiopaque along their length and particularly at their distal end such that the physician is able to clearly monitor the progress of the catheter as it is being advanced from the vascular access site to the vascular target site.

After the intravascular catheter has been navigated through the patient's vascular system with the distal end thereof adjacent the target site, the catheter may be used for various diagnostic and/or therapeutic purposes. Frequently, diagnostic and therapeutic techniques require the infusion of fluids through the catheter. For example, it may be desirable to inject radiopaque contrast media through the catheter to provide enhanced fluoroscopic visualization for diagnostic purposes, or to inject pharmaceutical solutions (i.e., drugs) to the target site for therapeutic purposes.

The blood vessels in the brain frequently have an inside diameter of less than 3 mm. Accordingly, it is desirable that intravascular catheters intended for use in these blood vessels have an outside diameter which allows the catheter to be easily accommodated by the blood vessel. The path of the vasculature inside the brain is highly tortuous, and the blood vessels are relatively fragile. Accordingly, it is desirable that the distal portion of a catheter be sized appropriately and be atraumatic for the neurological vasculature.

### SUMMARY OF THE INVENTION

The present invention comprises a unique intravascular catheter that incorporates a number of refinements to the shaft and distal tip. According to a preferred embodiment of the invention, a catheter comprises a shaft having a proximal end, a distal end, and a lumen. A hub is typically disposed at the proximal end and a distal tip is disposed at the distal end. The shaft may comprise multiple layers, including an inner liner, a second layer, a third layer, and a fourth layer.

The second layer may be disposed over the inner liner extending from the proximal end of the shaft to a distal terminus. The distal terminus may be about 4 millimeters from the distal end. The absence of the second layer between the distal terminus and the distal end of the shaft improves the physical properties of the catheter. For example, the shaft may be more flexible or generally softer near the distal end, and may be more readily thermoformed.

The third layer may be disposed over the second layer and preferably comprises a coil that is wound over the second layer. The coil may be arranged in a single coil region near the distal end of the shaft. The single coil region is understood to be a single layer of coil wound around the second layer along a longitudinal axis thereof. The coil may further include a multiple coil region near the proximal end of the shaft wherein the coil is wound multiple times around the second layer along the longitudinal axis thereof.

The fourth layer may be disposed over the third layer and may include a taper. Preferably, the taper decreases the diameter of the shaft near the distal end thereof. The decrease in diameter may comprise a suitable reduction in size appropriate for multiple uses of the catheter. For example, a generally small diameter distal tip may be used for procedures involving treatment of relatively small blood vessels.

### BRIEF DESCRIPTION OF THE DRAWINGS

FIG. 1 is a plan view of an intravascular catheter with an improved shaft, distal tip, and transitions according to a preferred embodiment of the invention;

FIG. 2 is an enlarged view of a shaft of the intravascular catheter shown in FIG. 1;

FIG. 3 is an enlarged view of an alternative shaft of the intravascular catheter shown in FIG. 1; and

FIG. 4 is an enlarged view of another alternative shaft of the intravascular catheter shown in FIG. 1.

### DETAILED DESCRIPTION OF THE PREFERRED EMBODIMENTS

The following description should be read with reference to the drawings wherein like reference numerals indicate like elements throughout the several views. The detailed description and drawings depict select embodiments and are not intended to be limiting.

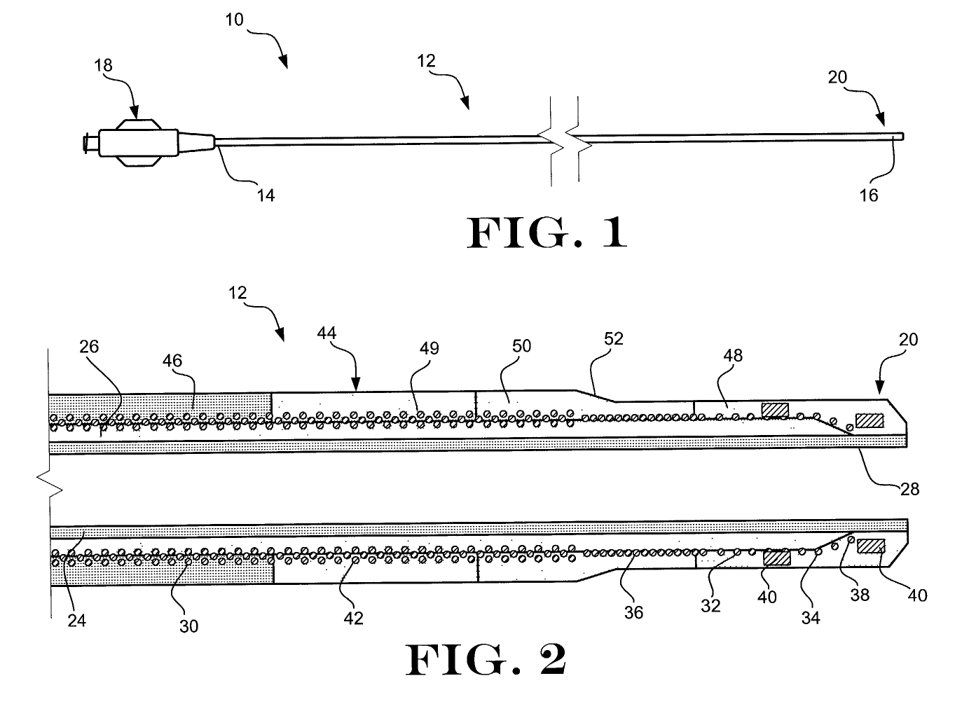

FIG. 1 is a plan view of an intravascular catheter 10 with an improved shaft, distal tip, and improved transitions according to a preferred embodiment of the invention.

The intravascular catheter 10 comprises a shaft 12 having a proximal end 14 and a distal end 16. A hub 18 is typically disposed at proximal end 14 of shaft 12 and a distal tip 20 having a shapable length is disposed at distal end 16 of shaft 12. Shaft 12 further comprises a lumen 22 as best seen in FIG. 2. Lumen 22 may be a guidewire lumen and/or an infusion lumen. Lumen 22 may have a diameter compatible with a guide wire having an outside diameter of about 0.010 to 0.014 inches.

Shaft 12 comprises multiple layers including an inner liner 24. Preferably, inner liner 24 comprises polytetrafluoroethylene (PTFE). Polytetrafluoroethylene is a preferred material because it creates a smooth, low-friction surface for the passage of other devices or fluids through catheter 10. In an alternate embodiment, inner liner 24 may comprise materials including, but not limited to, thermoplastics, high performance engineering resins, fluorinated ethylene propylene (FEP), polymer, polyethylene (PE), polypropylene (PP), polyvinylchloride (PVC), polyurethane, polyether-ether ketone (PEEK), polyimide, polyamide, polyphenylene sulfide (PPS), polyphenylene oxide (PPO), polysufone, nylon, or perfluoro(propyl vinyl ether) (PFA).

Inner liner 24 may be formed by extrusion over a mandrel. Extrusion may result in inner liner 24 having a thickness of about 0.0005 inches to 0.00125 inches and a diameter of about 0.0175 inches to 0.019 inches over a length of about 135 cm to 200 cm. In an alternate embodiment, inner liner 24 may be formed by lamination over a mandrel. The mandrel may, for example, comprise nitinol and have a diameter of about 0.0165 inches. A person of ordinary skill in the art would be familiar with processes and equipment suitable for forming inner liner 24 according to multiple embodiments of the present invention.

A second layer 26 is disposed over inner liner 24. Second layer 26 is comprised of polyether block amide (PEBA). Polyether block amide is commercially available from Atochem Polymers of Birdsboro, Pennsylvania, under the trade name PEBAX. Second layer 26 may comprise PEBAX 55 having a diameter of about 0.0185 inches to 0.022 inches and a length of about 132 cm to 200 cm.

Second layer 26 extends from proximal end 14 of shaft 12 to a distal terminus 28. Distal terminus 28 is set back from distal end 16 a distance that is equal to or greater than the shapable length of distal tip 20. For example, distal terminus 28 may be 4 millimeters to 3 centimeters from distal end 16 depending on the flexibility and shapable length desired. The absence of second layer 26 between distal terminus 28 and distal end 16 of shaft 12 improves the physical properties of catheter 10. For example, shaft 12 may be more flexible or generally softer near distal end 16, and/or may be more shapable by thermoforming techniques.

Second layer 26 may be formed by securing outer layer 26 near distal end 16 of shaft 12 and laminating to proximal end 14 thereof. Alternatively, second layer 26 may be disposed over inner liner 24 by extrusion.

A third layer 30 is disposed over second layer 26. Third layer 30 comprises a coil manufactured from materials including, but not limited to, stainless steel, metal, nickel alloy, nickel titanium alloy, polymer, round wire, flat wire, magnetic resonance imaging compatible metal, and combinations thereof. A magnetic resonance imaging compatible metal is understood to comprise non-magnetic or non-ferrous metals.

Third layer 30 further comprises a single coil region 32 near distal end 16. The coil may be wound around second layer 26 along a substantial portion of the length thereof. Single coil region 42 is understood to be a single layer of coil wound around second layer 26 along a longitudinal axis thereof, e.g., 0.0125 inch outside diameter stainless steel round wire. Third layer 30 further includes a multiple coil region 42 near proximal end 14 of shaft 12 wherein coil is wound multiple times around second layer 26 at a particular point along the longitudinal axis thereof.

Single coil region 32 further comprises a first pitch region 34 and a second pitch region 36. First pitch region 34 comprises a pitch between about 0.050 inches per turn and 0.004 inches per turn. Second pitch region 36 comprises a pitch between about 0.020 inches per turn and 0.002 inches per turn. Those skilled in the art will recognize that a number of values may be used to describe the pitch of first pitch region 34 and second pitch region 36 without deviating from the spirit and scope of the invention. For example, first pitch region 34 and second pitch region 36 may be substantially equal.

A distal end 38 of third layer 30 may be secured to a radiopaque marker 40. Preferably, radiopaque markers 40 produce a relatively bright image on a fluoroscopy screen during a medical procedure. This relatively bright image aids the user of catheter 10 in determining the location of distal end 16 of shaft 12. Radiopaque markers 40 may comprise a number of radiopaque materials including, but not limited to, gold, platinum, and plastic material loaded with a radiopaque filler. Catheter 10 may further comprise additional radiopaque markers.

A fourth layer 44 is disposed over third layer 30. Fourth layer 44 comprises polyether block amide (PEBA). Alternately, fourth layer 44 may be comprised of materials similar to those disclosed above, including polymers and metals. Fourth layer 44 may have a length of about 135 cm to 200 cm.

Fourth layer 44 further comprises a proximal end 46, a distal end 48, a first middle section 49, and a second middle section 50. Each individual section of fourth layer 44 may comprise polyether block amide. The durometer of each section may be different. At distal end 48, the preferred material is a low durometer polymer (e.g., PEBAX 2533) to maintain a soft, atraumatic tip. At proximal end 46, the preferred material is a high durometer polymer (e.g., PEBAX 7233) to provide pushability. First middle section 49 and second middle section 50 may provide a smooth transition between proximal end 46 and distal end 48. For example, first middle section 49 may comprise PEBAX 4033 and second middle section 50 may comprise PEBAX 5533. Generally, the durometer decreases from proximal end 46 to distal end 48. Alternatively, fourth layer 44 may be comprised of a single section having a differing durometer on opposite ends.

Fourth layer 44 further comprises a taper 52. Taper 52 decreases the diameter of shaft 12 near distal end 16. Taper 52 may decrease the diameter of shaft 12 to varying degrees. The outside diameter of fourth layer 44 may be about 0.026 inches to 0.035 inches near proximal end 46 and about 0.021 inches to 0.026 inches at distal end 48. Preferably, the outside diameter of shaft 12 from taper 52 to distal end 16 is sized appropriately for insertion into generally small blood vessels. For example, distal end 16 may be sized to facilitate entry of shaft 12 into the coronary, peripheral, and neurological vasculature.

Fourth layer 44 may be disposed over third layer 30 by heat fusing separate tube sections 46, 48, 49, and 50 by extrusion. Alternatively, fourth layer 44 is disposed over third layer 30 by lamination.

The combination of layers at distal end 16 of shaft 12 comprises a level of flexibility which makes it unlikely to damage the blood vessels of a patient. According to this embodiment, distal tip 20 is understood to comprise an atraumatic and shapable tip. Moreover, the shapable length of distal tip 20 can be heat set, for example by steam.

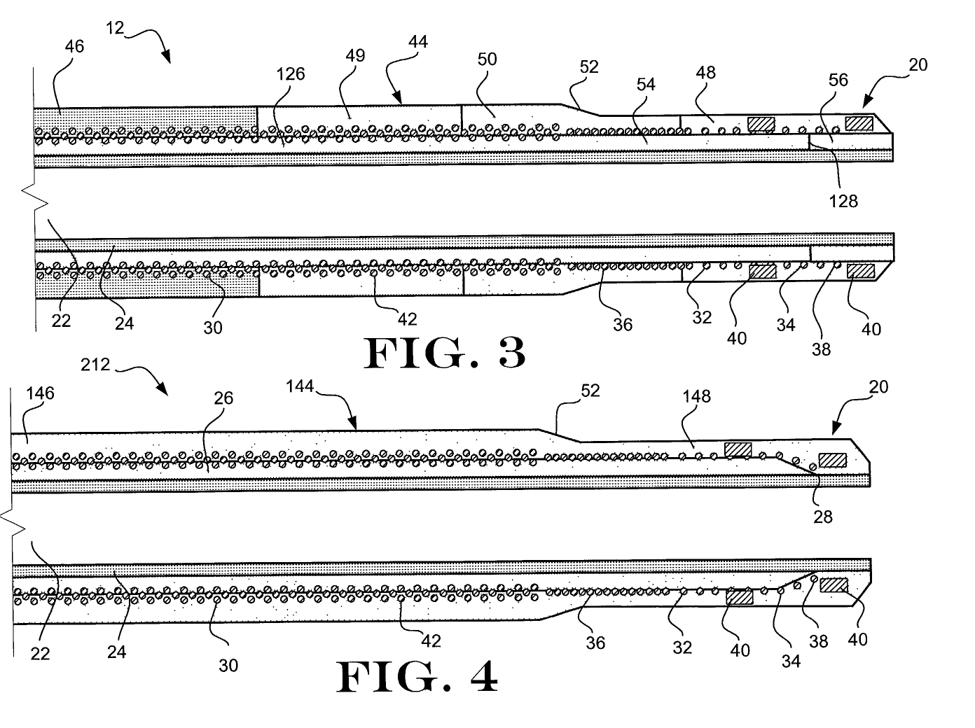

FIG. 3 is an enlarged view of an alternate shaft 112 that is essentially similar to shaft 12 with a refinement to second layer 26. Second layer 126 extends from proximal end 14 of shaft 112 to distal terminus 128. Second layer 126 further comprises a second segment 56. Preferably, first segment 54 extends from proximal end 14 of shaft 112 to distal terminus 128 and is substantially similar to second layer 26 as depicted in FIG. 2. Second segment 56 preferably extends from distal terminus 128 to distal end 16 of shaft 12. Distal terminus 128 is set back from distal end 16 of shaft 112 a distance equal to or greater than the shapable length of distal tip 20. The durometer of first segment 54 and second segment 56 are different. For example, first segment 54 comprises a generally harder durometer (e.g., PEBAX 5533D) than second segment 56 (e.g. PEBAX 2533D).

Shaft 112 may be manufactured substantially similar to what is disclosed above for shaft 12. A person of ordinary skill in the art would be familiar with alterations in the method of manufacture according to multiple embodiments of the invention.

FIG. 4 is an enlarged view of an alternative shaft 212 that is essentially similar to shaft 12 with a refinement to fourth layer 44. Fourth layer 144 is disposed over third layer 30. Fourth layer 144 further comprises proximal end 146 and distal end 148. Preferably, fourth layer 144 is comprised of a single layer of PEBA having a differing durometer on opposite ends. For example, the durometer of proximal end 146 may be greater than the durometer of distal end 148. Fourth layer 144 can be disposed over third layer 30 by gradient extrusion. Gradient extrusion is described in U.S. patent application Ser. No. 09/430,327 to Centell et al., which is hereby incorporated by reference. In summary, gradient extrusion is understood to be an extrusion technique wherein polymers of differing durometer may be disposed onto an object so as to form a smooth transition in a physical property (e.g., durometer). For example, gradient diffusion of fourth layer 144 may result in a generally harder durometer (e.g., PEBAX 7233) near proximal end 146 and a generally softer durometer (e.g., PEBAX 2533) near distal end 148. In addition, gradient diffusion of fourth layer 144 would result in a substantially gradual decrease in durometer from proximal end 146 to distal end 148.

In a preferred embodiment, shaft 212 may be manufactured substantially similar to what is disclosed above for shaft 12. A person of ordinary skill in the art would be familiar with alterations in the method of manufacture according to multiple embodiments of the invention.

Numerous advantages of the invention covered by this document have been set forth in the foregoing description. It will be understood, however, that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An intravascular catheter, comprising:
an elongate shaft having a proximal end, a distal end, and a distal tip, the elongate shaft including:
an inner liner;
a second layer disposed over the inner liner, the second layer extending from the proximal end of the shaft to a distal terminus;
a third layer disposed over the second layer; and
a fourth layer disposed over the third layer, the fourth layer including a proximal end and a distal end.

2. The catheter in accordance with claim 1, wherein the distal tip has a shapable length, and wherein the distal terminus is set back from the distal end of the shaft a distance equal to or greater than the shapable length.

3. The catheter in accordance with claim 2, wherein the distal terminus is about 4 millimeters from the distal end of the shaft.

4. The catheter in accordance with claim 3, wherein the shape of the distal tip can be heat set.

5. The catheter in accordance with claim 4, wherein the shape of the distal tip can be heat set by steam.

6. The catheter in accordance with claim 3, wherein the inner liner comprises polytetrafluoroethylene.

7. The catheter in accordance with claim 3, wherein the second layer comprises polyether block amide.

8. The catheter in accordance with claim 3, wherein the third layer comprises a coil.

9. The catheter in accordance with claim 8, wherein the coil comprises stainless steel.

10. The catheter in accordance with claim 8, wherein the coil comprises nickel alloy.

11. The catheter in accordance with claim 8, wherein the coil comprises a non-ferrous metal.

12. The catheter in accordance with claim 3, wherein the fourth layer comprises polyether block amide.

13. The catheter in accordance with claim 3, wherein the distal end of the shaft has an outside diameter that is less than the outside diameter of the proximal end of the shaft.

14. The catheter in accordance with claim 3, wherein the distal end of the shaft has a durometer that is less than that of the proximal end of the shaft.

15. The catheter in accordance with claim 3, further comprising a radiopaque marker.

16. The catheter in accordance with claim 15, wherein the distal end of the third layer is secured by the radiopaque marker.

17. The catheter in accordance with claim 3, wherein the second layer further comprises a second segment.

18. The catheter in accordance with claim 17, wherein the second segment is disposed at the inner liner between the distal terminus and the distal end of the shaft.

19. An intravascular catheter, comprising:
an elongate shaft having a proximal end, a distal end, and a distal tip having a shapable length, the elongate shaft including:
an inner liner;
a second layer disposed over the inner liner, the second layer extending from the proximal end of the shaft to a distal terminus, wherein the distal terminus is set back from the distal end of the shaft a distance equal to or greater than the shapable length;
a third layer disposed over the second layer; the third layer including a single coil region near the distal end of the shaft and a multiple coil region near the proximal end of the shaft; and
a fourth layer disposed over the third layer, the fourth layer including a proximal end and a distal end, wherein the durometer at the proximal end is greater than the durometer at the distal end.

20. The catheter in accordance with claim 19, wherein the distal terminus is about 4 millimeters from the distal end of the shaft.

21. The catheter in accordance with claim 20, wherein the shape of the distal tip can be heat set.

22. The catheter in accordance with claim 21, wherein the shape of the distal tip can be heat set by steam.

23. The catheter in accordance with claim 21, wherein the inner liner comprises polytetrafluoroethylene.

24. The catheter in accordance with claim 21, wherein the second layer comprises polyether block amide.

25. The catheter in accordance with claim 21, wherein the third layer comprises a coil.

26. The catheter in accordance with claim 25, wherein the coil comprises stainless steel.

27. The catheter in accordance with claim 25, wherein the coil comprises nickel alloy.

28. The catheter in accordance with claim 25, wherein the third layer comprises a non-ferrous metal.

29. The catheter in accordance with claim 21, wherein the fourth layer comprises polyether block amide.

30. The catheter in accordance with claim 21, wherein the distal end of the shaft has an outside diameter that is less than the outside diameter of the proximal end of the shaft.

31. The catheter in accordance with claim 21, further comprising a radiopaque marker.

32. The catheter in accordance with claim 31, wherein a distal end of the third layer is secured by the radiopaque marker.

33. The catheter in accordance with claim 21, wherein the second layer further comprises a second segment.

34. The catheter in accordance with claim 33, wherein the second segment is disposed at the inner liner between the distal terminus and the distal end of the shaft.

35. A method for manufacturing a catheter, comprising the steps of:
providing a mandrel;
extruding an inner liner over the mandrel, the inner liner defining a shaft having a proximal end, a distal end, and a distal tip that is shapable;
laminating a second layer over the inner liner from a distal terminus to the proximal end; wherein the distal terminus is set back from the distal end of the shaft a distance equal to or greater than the shapable length;
disposing a third layer over the second layer;
extruding a fourth layer over the third layer, the fourth layer including a proximal end and a distal end, wherein the durometer at the proximal end is greater than the durometer at the second end; and
removing the mandrel.

36. The method in accordance with claim 35, wherein the step of disposing a second layer over the inner liner further comprises disposing a second segment over the inner liner.

37. The method in accordance with claim 35, wherein the step of extruding a fourth layer over the third layer further comprises forming a taper within the fourth layer wherein the distal end of the shaft has an outside diameter that is less than the outside diameter of the proximal end of the shaft.

38. The method in accordance with claim 35, wherein the step of extruding a fourth layer over the third layer includes gradient extrusion.

39. The method in accordance with claim 35, wherein the distal end of the third layer is secured by a radiopaque marker.

40. The method in accordance with claim 35, wherein the step of laminating a second layer of the inner liner further comprises disposing a second segment on the inner liner between the distal terminus and the distal end of the shaft.

41. An intravascular catheter, comprising:
an elongate shaft having a proximal end, a distal end, and a distal tip, the elongate shaft including:
an inner liner;
a second layer disposed over the inner liner, the second layer extending from the proximal end of the shaft to a distal terminus;
a second segment extending from the distal terminus to the proximal end;
a third layer disposed over the second layer; and
a fourth layer disposed over the third layer, the fourth layer including a proximal end and a distal end.
